(19) United States
(12) Patent Application Publication
(10) Pub. No.: US 2015/0216722 A1
(43) Pub. Date: Aug. 6, 2015

(54) METHOD AND APPARATUS FOR ULTRASONIC EYE CLEANER

(71) Applicant: John R. CHOATE, Milford, MI (US)

(72) Inventor: John R. CHOATE, Milford, MI (US)

(21) Appl. No.: 14/174,608

(22) Filed: Feb. 6, 2014

(51) Int. Cl. A61F 9/00

(52) U.S. Cl. CPC A61F 9/00

(57) ABSTRACT

An ultrasonic eye cleaning device comprises a body having an internal cavity and an end. The device further comprises a motor within the internal cavity, wherein the motor has a rotating output member. The device further comprises a chuck driven by the rotating output member of the motor. The device further comprises a piezo driver cooperating with the body and at least one of the rotating output member or the chuck to oscillate the chuck at 20 kHz to 100 MHz. The device further comprises an instrument that is attachable to the chuck and defining a free end forming a cleaning swab.

### TECHNICAL FIELD

The present invention relates to an ultrasonic eye cleaning device, and more particularly, to an impedance transmission tip for ultrasonic vibration to prevent eyelid marginal disease and for treating an ocular disorder.

### BACKGROUND

Ocular disorders such as those relating to eyelid margin disease are particularly common pathological conditions of the ocular adenexa. By way of example, these disorders include blepharitis, meibomitis, meibomian glad dysfunction, and dry eye syndrome. Despite advances in ophthalmology and medical treatments in general, the recommended treatments for these exemplary common ocular disorders has remained essentially unchanged for decades.

One common ophthalmologic cause relating to dry eye disorder is known as meibomian gland dysfunction. The meibomian gland (also known as the tarsal gland) is located inside the tarsal plate at the rim of the eyelids. The meibomian gland is a sebaceous gland that is responsible for the supply of meibum that prevents the tear film from evaporating and is an oily substance. Meibum allows the eye to close which blocks the tear fluid between the edge of the eyelid and the eyeball, and prevents tears from falling onto the cheek. There are approximately 50 meibomian glands located on the upper eyelids and 25 on the lower eyelids.

When the meibomian gland fails to perform it causes dry eye. Dysfunction of these glands cause tears to evaporate more rapidly and leads to symptoms of dryness, burning, and irritation. There are naturally occurring bacteria that thrive on the corneal surface that can colonize on the eyelid and the meibomian glands and cause problems. This failure can lead to blepharitis, which is an inflammation of eyelid skin. When the meibomian gland swells it leads to a condition called meibomitis which is identified by a thick, waxy secretion from the obstructed gland.

The primary cause for this dysfunction of the glands is that they get impacted and clogged. The thickening of these oils in addition to the increased populations of bacteria can gradually decrease the secretion of desired fluids (e.g., oils) from the glands.

Additionally, blepharitis caused by microscopic ectoparasite infestations (Demodex—which are found in the human skin) is one of the leading causes of eyelid inflammation. The rate of Demodex infestation increases with age, being observed in 84% of the population at age 60 and in 100% of those older than 70 years. Apart from its higher density in patients with rosacea, Demodex mites have also been suggested as a cause of other skin diseases such as pityriasis folliculorum, perioral dermatitis, scabies-like eruptions, facial pigmentation, eruptions of the bald scalp, demodicosis gravis, and even basal cell carcinoma. Because the eye is surrounded by such protruding body parts as the nose, the brow, and the cheek, the eyelid is not as accessible as the face to daily cleansing hygiene. Therefore, once Demodex infestation establishes in the face, it is likely to spread and flourish in the eyelids leading to blepharitis. Demodex blepharitis is often overlooked in differential diagnosis of corneal and external diseases.

Two distinct species of Demodex have been identified in humans: D. folliculorum and D. brevis. In the eyelids, D. folliculorum can be found in the lash follicle, whereas D. brevis burrows deep into sebaceous glands and meibomian glands looking for sebum, which is thought to be their main food source. It has also been proposed that these mites might feed on follicular and glandular epithelial cells leading to direct damage of the lid margin. The life cycle of the Demodex mite is approximately 14-18 days from the egg to the larval stage followed by 5 days in the adult stage. Females may live an additional 5 days after oviposition. Because of the limited life span of the adult mites, mating plays an important role in perpetuating Demodex infestation. Furthermore, Demodex's life span is limited outside the living body, thus, direct contact is required for transmission of mites. Accordingly, it is important to prevent mating of mites and avoid direct transmission as an indispensable treatment strategy.

Historically, treatment of eyelid margin disease begins and ends with the patient. The patient first begins to notice symptoms including eyelid margins, and a gritty sensation of the eye culminating in inflammation, irritation, burning, and reduced vision. Should these symptoms remain unchanged or worsen, the patient routinely seeks the advice of an eye specialist, such as an ophthalmologist or optometrist. After carefully considering the patients' medical history and investigating various possible causes, the specialist may prescribe a hygienic home treatment procedure for the patient to perform regularly in conjunction with antibiotics and/or topical steroids until the disease subsides.

The hygienic home treatment procedure is to remove debris, oil, and scurf that have collected along the eyelid margin during progression of the disorder. Removal of this debris is critical to both healing the eye and preventing a resurgence of the disorder. Without proper, regular removal of accumulated debris, such ocular disorders regularly worsen despite periodic treatments.

The hygienic home treatment of such ocular disorders is generally a two-step process. First, the patient softens the debris and scurf by applying a warm compress, diluted baby shampoo, or a specialized liquid solution to the eyelid margin. This first step is intended to prepare the debris for removal while preventing further irritation to the eye. The second step attempts to remove the debris by physically scrubbing the eyelid margin, the base of the eyelashes, and the pores of the meibomian glands. This scrubbing is routinely attempted with either a generic cotton swab, a fingertip, or a scrub pad placed over the fingertip and applied against the eye. By cleaning debris and scuff free from the base of the eyelashes and unclogging the pores of the meibomian glands, the patient may improve the overall health of the eyelid margin; thereby reducing irritation, burning, and other symptoms related to the disorder.

The hygienic home treatment is met with limited success due to the practical difficulties of cleaning one's own eye with an imprecise instrument such as a fingertip or cotton swap. For instance, many patients do not have the necessary dexterity to manipulate their fingertip or a cotton swab along the eyelid margin. Moreover, a shake, tremor, or poor near vision further complicate such self-treatment. Even for those capable of incorporating hygienic home treatment into their daily routine, many, if not most people, are wary of placing objects near their eyes to actively scrub along the eyelid margin. Given this anxiety, discomfort, and the inability to specifically target debris deposits, patients routinely fail to totally cleanse the margin of the eyelid, the base of the eyelashes, and the openings of the meibomian glands. While the attempted treatment may temporarily abate the patient's symptoms, subtle continuation of the disease often persists; thus permitting a low-grade inflammation to develop and, ultimately lead to chronic dry eye syndrome. Further, this treatment is typically required to be performed for the rest of the patient's life; thereby, creating a substantial hurdle to regular and effective compliance during hygienic home treatment.

Evidence suggests that medical costs associated with dry eye syndrome, often induced by ocular diseases such as blepharitis, are currently over 68 billion dollars each year. Many of these expenses are needlessly incurred due to the patients' failure to perform regular and effective treatments resulting in increased doctor visits, medications, and artificial tears. These expenses create a significant financial burden for insurance carriers, especially Medicare, which provides primary medical coverage for many individual particularly prone to dry eye disease, such as the elderly.

There is a need for a method and apparatus for use in treating ocular disorders and preventing eyelid marginal diseases, that addresses present challenges and characteristics such as those discussed above.

### SUMMARY

In a first illustrative embodiment, an ultrasonic eye cleaning device comprising a body having an internal cavity and an end. The device further comprising a motor within the internal cavity, wherein the motor has a rotating output member. The device further comprising a chuck driven by the rotating output member of the motor. The device further comprising a piezo driver cooperating with the body and at least one of the rotating output member or the chuck to oscillate the chuck at 20 kHz to 100 MHz. The device further comprising an instrument having a tip that is attachable to the chuck and defining a free end forming a cleaning swab.

In a second illustrative embodiment, an eye cleaning device comprising an elongate body having an internal cavity and an open end. The device further comprises an output member having a base provided within the internal cavity and a shaft adjacent to the open end of the body. The device further comprising a piezoelectric driver within the internal cavity and attached to the base of the output member. The piezoelectric driver, when energized, oscillates the output member at 20 kHz to 100 MHz. The device further comprising an instrument positioned at the open end of the body and attached to the shaft of the output member. The device further comprising a pad attached to the instrument to be oscillated by the piezoelectric driver.

In a third illustrative embodiment, an eye cleaning method to treat an eye having an ocular disorder using an eye cleaning device. The method may generate an ultrasonic oscillation of an instrument having a free end forming a cleaning swab. The instrument may be affixed to the ultrasonic eye cleaning device and provides the ultrasonic oscillation to the free end causing the cleaning swab to oscillate from 20 kHz to 100 MHz. The method may move the instrument relative to the ultrasonic eye cleaning device. While the cleaning swab is being moved by the ultrasonic eye cleaning device, the method contains a portion of the eye that includes debris based on the ocular disorder with the cleaning swab thereby impacting the debris with the ultrasonic oscillation with the swab and removing the debris from the eye.

### BRIEF DESCRIPTION OF THE DRAWINGS

FIG. 1 depicts a drawing of an ultrasonic eye cleaning device according to an embodiment;

FIG. 2 depicts a drawing of a user interface for the ultrasonic eye cleaning device according to an embodiment;

FIG. 3 depicts a drawing of a configuration of the piezoelectric driver cooperating with an electric motor within the body of the ultrasonic eye cleaning device according to an embodiment;

FIG. 4 depicts a drawing of a configuration of the piezoelectric driver cooperating with the electric motor within the body of the ultrasonic eye cleaning device according to an embodiment;

FIG. 5 depicts a drawing of a configuration of the piezoelectric configured with the electric motor within the body of the ultrasonic eye cleaning device according to an embodiment;

FIG. 6 depicts a drawing of a configuration of the piezoelectric within the body of the ultrasonic eye cleaning device according to an embodiment;

FIG. 7A-7C depict drawings of an instrument having at least one of a rotating and oscillating tip of the ultrasonic eye cleaning device and a disposable cleaning swab according to an embodiment; and

FIG. 8A-8C depict drawings of the device used in a method for treating ocular disorders of an eye.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

The embodiments of the present disclosure generally provide for a plurality of circuits or other electrical devices. All references to the circuits and other electrical devices and the functionality provided by each, are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microprocessors, integrated circuits, memory devices (e.g., FLASH, random access memory (RAM), read only memory (ROM), electrically programmable read only memory (EPROM), electrically erasable programmable read only memory (EEPROM), or other suitable variants thereof) and software which co-act with one another to perform operation(s) disclosed herein. In addition, any one or more of the electric devices may be configured to execute a computer-program that is embodied in a non-transitory computer readable medium that is programmed to perform any number of the functions as disclosed.

The embodiments of the present disclosure is to provide an ultrasonic eye cleaning device using a piezoelectric ultrasonic motor arrangement that may include piezoelectric crystals configured with and without an electric motor. The ultrasonic eye cleaning device provides ultrasonic vibration that may be effectively transmitted to a tip portion of the device configured with a fixed wand instrument, a swab, or a removable cell foam tip. The ultrasonic vibration at the tip of the eye cleaning device may provide the ability to break up scales on the skin while emitting resonate sound waves into the skin creating a cavitation effect. The ultrasonic vibration at the swab tip may also provide a more effective delivery for the treatment of the eyelid marginal disease and/or for treating an ocular disorder by creating a sonophoresis effect to allow direct targeting of a bioactive compound to the treatment area of the eyelid. For the ultrasonic eye cleaning device described herein, the method and apparatus may be described in further detail below.

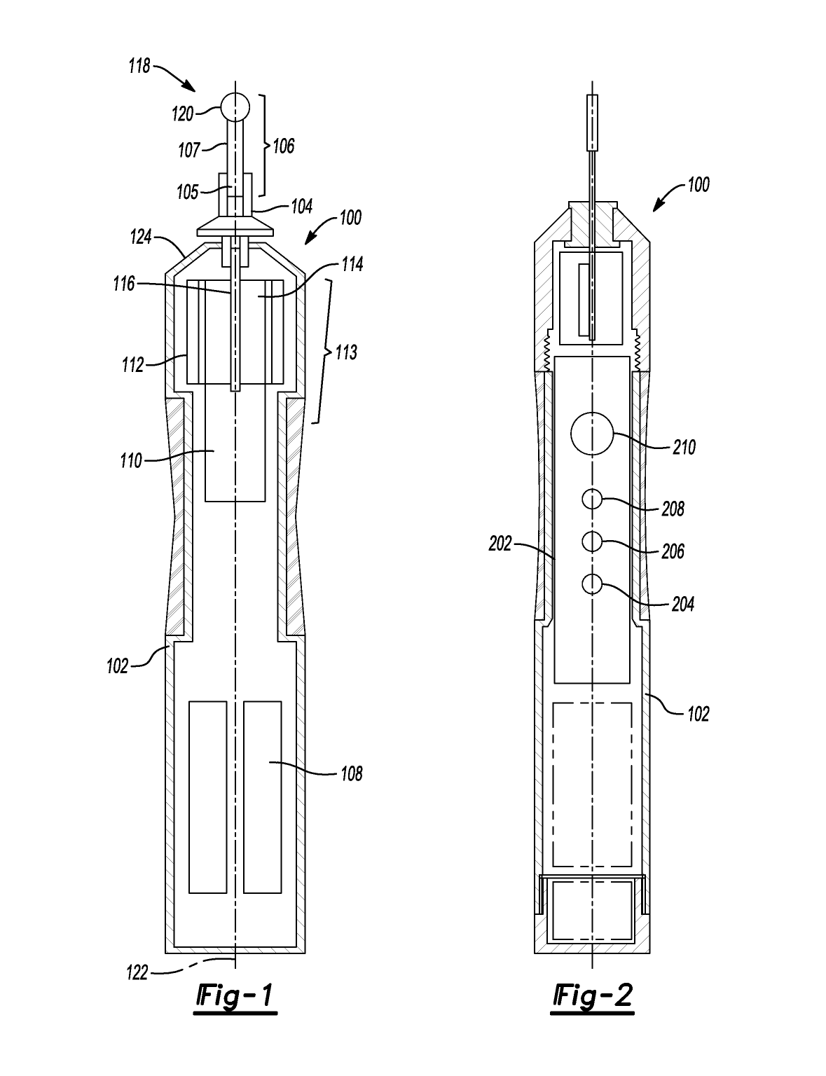

FIG. 1 depicts a drawing of an ultrasonic eye cleaning device 100 according to an embodiment. The eye cleaning device 100 includes a body 102 which houses a drive unit system 113 that operably moves an output member 116 in communication with an instrument 106 to facilitate removal of debris from an eye. The drive unit system 113 may include, but is not limited to, a piezo ceramic tube 112 attached to an outer housing of a motor 114. The drive unit system 113 may be controlled by one or more circuits configured on a printed circuit board 110. The drive unit system 113 may receive power from an electric power source 108 to enable the device 100 to a power on state.

The electric power source 108 is a battery power source contained within the body 102 of the device 100 or may be powered via an outside power source (not shown) delivered by a cable to the device 100. The electronic power source 108 may include, but is not limited to, a battery power source that is either disposable or rechargeable. The electric power source 108 operably provides electrical power to the electric motor 114 and the piezo ceramic tube 112, which the operator controls via a user interface having a control switch which will be described further in FIG. 2.

The instrument 106 may be fixed or removable. In this embodiment, the instrument 106 is changeable and is connected to a removable compression fit sleeve herein known as a chuck 104. The chuck 104 having a distal end and a proximal end portion. The instrument 106 is attached to the distal end of the chuck 104, which is configured to operate with the drive unit system 113. The proximal end portion of the chuck 104 may be removably secured to the drive unit system 113 in order to transmit motion from the drive unit, through the output member 116, to the instrument 106.

The instrument 106 may include a tip 120. The tip may be used as a cleaning swab to remove debris on an eye. The cleaning swab may include, but is not limited to, an open cell PU foam, a closed cell foam, or any other suitable material used to remove debris on the eye. The instrument 106 may be a size sufficient to access debris on the eye and eyelid area while transmitting the ultrasonic frequency to the tip 120. For example, the instrument may have an approximate length of 10-45 millimeters, and an approximate width of 5-30 millimeters. The tip 120 may be a size sufficient to access debris on the eye and eyelid area while receiving the ultrasonic oscillation to break apart debris on the eye. For example, the tip 120 may have an approximate length between 1.0-30 millimeters and an approximate width of 5-30 millimeters. More particularly, the tip 120 has an approximate length of 8 millimeters and an approximate width of 5-10 millimeter. It may be appreciated that the tip 120 may be manufactured of any material suitable for contacting the eye without harming the eye. The tip 120 as shown in FIG. 1 may include a sponge. The sponge may include any material that is soft, porous, and resilient. The tip 120 may include, but is not limited to, a medical grade sponge or a surgical grade sponge capable of removing debris on an eye and eyelid area without harming the eye.

In another embodiment, the instrument 106 may be affixed to the body 102 of the device 100. The fixed instrument 106 may be configured with the tip 120. The fixed instrument 106 may be configured to receive a disposable instrument cover (e.g., an open cell foam tip). The disposable instrument cover may include any material that is soft, porous, and resilient while fitting the form of the instrument. The disposable instrument cover may mimic the movement of the instrument 106 and/or tip 120 for removing/breaking debris on the eye. The disposable instrument cover will be described in further detail below.

The instrument 106 may transmit motion from the drive unit system 113 to the tip 120. The instrument 106 may include a cylindrical shaft 107 having a central access 122. The shaft 107 extends along the central axis 122 between the handle 102 and the tip portion 120. The shaft 107 is sufficiently rigid to effectively transmit motion from the drive unit system 113 to the tip portion 120. The tip portion 120 is permanently affixed to the instrument 106 by forming a base portion to the shaft 107 during manufacturing. However, it will be appreciated that any known method of affixing the tip portion 120 to the shaft 107 may be used. The shaft 107 may include any material or shape so long as the instrument 106 is rigid enough to transmit sufficient motion from the drive unit system 113 to the tip portion 120 in order to oscillate based on the ultrasonic frequency to break apart the debris and remove debris from the eye or on the eyelid area.

The drive unit system 113 configuration having the piezo ceramic tube 112 and the electric motor 114 may allow movement 118 of the instrument 106 and the tip portion 120. The movement 118 generated by the chuck 104 may include at least one of a rotational motion, a vibrating motion, an axial oscillation motion, and a reciprocating motion. The piezo ceramic tube 112 may generate ultrasonic frequencies at the tip 120 producing cavitation and/or sonophoresis that may assist in minimizing the over growth of bacteria and the endotoxins that the bacteria produces on the eyelid and lashes.

The axial oscillation motion may be either along the central axis 122 of the shaft 107 or orthogonal to the central axis 122 of the shaft 107. The vibrating motion may include ultrasonic motion generated by the piezo ceramic tube 112. The ultrasonic motion may include a frequency range from sonic frequencies to ultrasonic frequencies. The rotational motion may include a speed of movement 118 of the tip portion 120 that is sufficient to remove debris from on the eye. The rotational motion is generated by the electric motor 114. The vibrating, rotating, reciprocating, and/or a combination thereof, may provide movement 118 of the tip portion 120 to break-away and remove debris from the eye.

For example, the ultrasonic eye cleaning device 100 may transmit a range of frequencies (e.g., 20 kHz to 100 MHz) either through a spinning tip 120 and/or a fixed instrument 106 having an oscillating tip 120 configuration to exfoliate the eyelid. The ultrasonic motion delivered by the piezoelectric driver (i.e., as shown as the piezo ceramic tube 112) may decrease the number of bacteria on the eyelid and eyelashes as well as decreasing the number of any living organisms that may reside on the eyelid or in the eyelash follicle. The frequency of low ultrasonic energy may spin water molecules across the skin in fast, powerful, noninvasive vibrations that may exfoliate the eyelids and eyelashes while eliminating bacteria. The ultrasonic energy may eliminate bacteria in the eyelid by penetrating the bacteria's membrane and therefore reducing the production of endotoxins which may contribute to the cause of inflammation. The ultrasonic energy may eliminate viruses in the eyelid by opening tiny pathways around cells with the non-invasive vibrations of the tip 120 and/or instrument 106 while reducing inflammation in the eyelid.

The drive unit system 113 including the electric motor 114 and piezo ceramic tube 112 are positioned within the cavity of the handle 102. The chuck 104 is operably connected to the drive unit system 113 at a forward end portion 124 of the handle 102. The proximal end portion 105 of the shaft 107 is removably secured to the chuck 104. The chuck 104 is generally any element capable of removably securing the shaft 107 to the drive unit system 113. The chuck 104 may be configured to tightened or loosened to respectively secure or remove the instrument 106 to the chuck 104. The operable connection of the electric motor 114 configured with the piezo ceramic tube 112 transmits a movement 118 through the chuck 104 to the instrument 106. The movement 118 is any motion relative to the drive unit system 113 or, more particularly, to the handle 102, that creates relative motion to the debris on the eye such that upon contacting the debris with the tip 120, the debris is removed.

FIG. 2 depicts a drawing of a user interface 202 for the ultrasonic eye cleaning device 100 according to an embodiment. The user interface 202 provides selectable options for the operation of the ultrasonic eye cleaning device 100. For example, the speed, rotating direction, and/or vibrating motion of the instrument 106 and/or tip 120 may be variable or otherwise selectable by an operator selecting a switch at the user interface 202. The operator of the device 100 may select a desirable speed/vibration and/or a forward or reverse direction via the user interface 202.

The user interface 202 is operably connected to the one or more circuits configured on a printed circuit board 110. The user interface 202 may include, but is not limited to, a multi-selector switch 210, a spin direction indicator 208, a speed/vibration indicator 206, and/or a status of the electric power source indicator 204. The multi-selector switch 210 may allow the operator to turn on the device 100 with a push of a button. The multi-selector switch 210 may control the movement 118 of the instrument 106/tip 120 based on the number of times the operator pushes the switch. For example, the operator may want the device at a slow rotational speed, and/or a slow vibrating speed, and/or a reverse rotation, therefore may push the multi-selector switch 210 a few times until the desired setting selection is reached.

The indicators may include, but is not limited to, a light-emitting diode (LED). The LED indicators may provide information to the operator. For example, the speed/vibration indicator 206 may notify the operator of a speed and/or frequency the tip portion 120 is set at based on one or more color indicators from the LED. In another example, the status of the electric power source indicator 204 may notify the operator of the charge status of the battery source based on a color indicator of the LED (e.g., the LED may be red to indicate battery power low or green to indicate a fully charged battery).

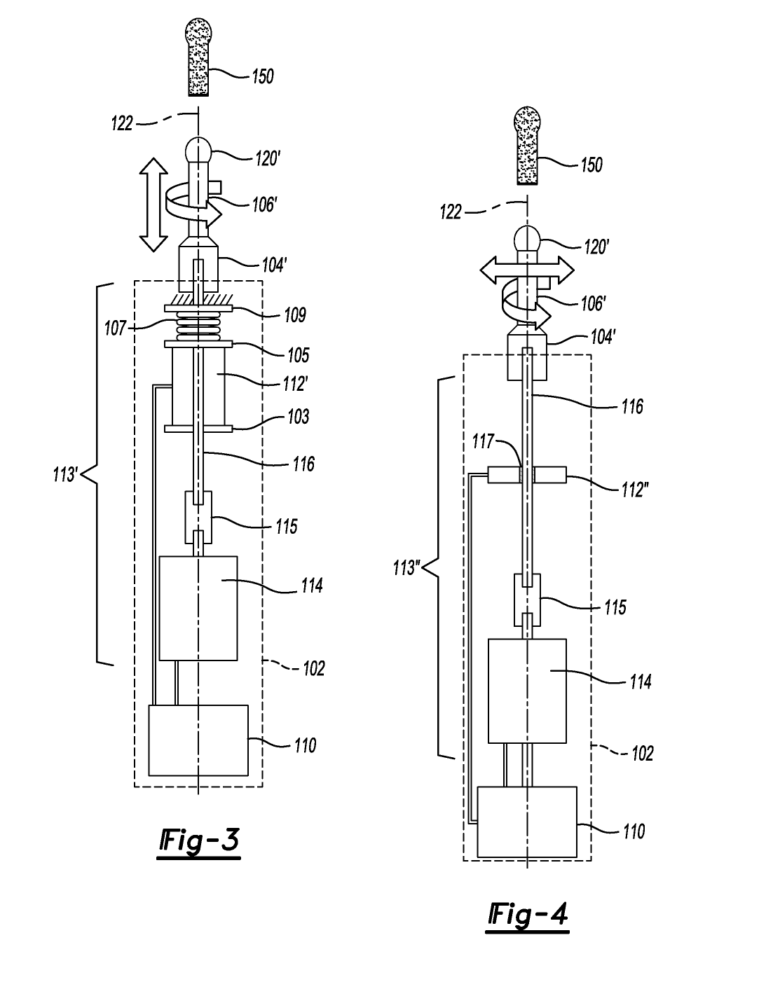

FIG. 3 depicts a drawing of a configuration of the piezoelectric cylinder 112′ cooperating with the electric motor 114 within the body 102 of the ultrasonic eye cleaning device 100 according to an embodiment. The configuration of the piezoelectric cylinder 112′ with the electric motor 114 may allow the chuck 104 to transmit motion through the instrument 106′ to allow the tip portion 120′ to move in an axial oscillation while rotating.

The device 100 may include a drive unit system 113′ comprising an electric motor 114 having a output member 116 connected to a shaft coupling 115. The shaft coupling is provided in the drive unit system 113′ to allow the output member 116 to transmit oscillating motion generated by the piezoelectric cylinder 112′ without producing shock loads to the electric motor 114. The piezoelectric cylinder 112′ is attached to the output member 116 configured with a fixed stop 103 at the base of the piezoelectric cylinder 112′, and a thrust washer 105 assembled with a spring 107 and a stop spring 109 above the cylinder 112′.

The drive unit system 113′ is configured with the electric motor 114 and the piezoelectric cylinder 112′ attached to the mechanical output 116 to generate an axial oscillation along the central axis 122 of the instrument 106′. The drive unit system 113′ may also produce a rotational motion of the tip portion 120′ while generating the axial oscillation along the central axis 122 of the instrument 106′. The device 100 may contain controls 110 comprising one or more circuits to receive an operator request to provide motion to the tip 120′ using the piezoelectric cylinder 112′ and/or the electric motor 114. The operator request may be received from the one or more selectable options proved at the user interface 202.

The drive unit system 113′ configuration as shown in FIG. 3 may provide an ultrasonic eye cleaning device 100 capable of treating various eye infection and/or diseases including, but not limited to, Blepharitis, Meibomian Gland Dysfunction, Chalazions, and/or Cicatricial Ectropion. For example, the device 100 is capable of treating blepharitis by breaking away the debris on the eyelid with the use of the axial oscillation of the tip 120′ while removing the debris with the rotational motion (e.g., forward and/or reverse direction) of the tip 120′. The device 100 may also treat blepharitis with the use of ultrasonic frequencies producing cavitation and/or sonophoresis that may assist in minimizing the over growth of bacteria and the endotoxins that the bacteria produces on the eyelid and eyelashes, as well as decreasing the number of any living organisms that may reside on the eyelid or in the eyelash follicle.

The device 100 may generate ultrasonic sound waves causing cavitation by opening up tiny pores in the thin upper layer of the skin, which then becomes more permeable. The device 100 may provide topical sonophoresis delivery of bioactive compounds thus allowing direct targeting to the skin with increased efficacy of absorption (e.g., 4,000-10,000 times more of an increase in the absorption rate) depending on the molecular weight of the active ingredient.

The device 100 may include a removable instrument 106 or a fixed stainless steel or titanium instrument 106′ that provides the movement generated by the drive unit system 113′. The fixed instrument 106′ may be configured to allow for the disposable instrument cover, herein known as a removable open cell foam tip 150, to be used for treatment while providing the simulated motions onto the eye, eye margins, and eyelid areas. The removable open cell foam tip may act as a cleaning swab to remove debris on an eye. The removable open cell foam tip 150 may be applied by covering over the tip 120′ and shaft 107 of the fixed instrument 106′. The removable open cell foam tip 150 allows an operator to dispose of the foam tip 150 after use. It may be appreciated that the removable open cell foam tip 150 may be manufactured of any material suitable for contacting the eye without harming the eye. The open cell foam tip 150 allows the ultrasonic frequency (e.g. sound waves) to pass through such that the treated area of the eye may receive the sounds waves.

FIG. 4 depicts a drawing of the configuration of a piezoelectric member 112″ cooperating with the electric motor 114 within the body 102 of the ultrasonic eye cleaning device 100 according to an embodiment. The drive unit system 113″ configuration of the piezoelectric member 112″ with the electric motor 114 may allow the chuck 104 to transmit motion through the instrument 106′ to allow the tip portion 120′ to move in a transverse oscillation along the center axis 122 while rotating.

The device 100 may include a drive unit system 113″ comprising the piezoelectric member 112″ configured with a bearing assembly 117 around the output member 116. The piezoelectric member 112″ may be configured on each side of the bearing assembly 117 such that it generates a transverse oscillation of the output member 116.

The drive unit system 113″ may also produce a motion at the tip portion 120′ that includes at least one of a rotational motion, a transverse oscillation motion (e.g., lateral oscillation) along the center axis 122, and a combination thereof. The piezoelectric member 112″ and the electric motor 114 may generate rotation and/or oscillation of the instrument 106′ based the controls 110 receiving operator input at the user interface 202.

The transverse oscillation motion generated by the piezoelectric member 112″ may provide a gentle frequency of ultrasonic energy vibrations that may exfoliate the eyelids and eyelashes, kill bacteria, decrease the number of any living organisms that may reside on the eyelid or in the eyelash follicle, and/or reduce inflammation of the treated area by the creation of cavitation. The transverse oscillation motion and/or rotational motion of the tip portion 120′ may break up and remove the debris on the eye, eye margins, and/or eyelid area.

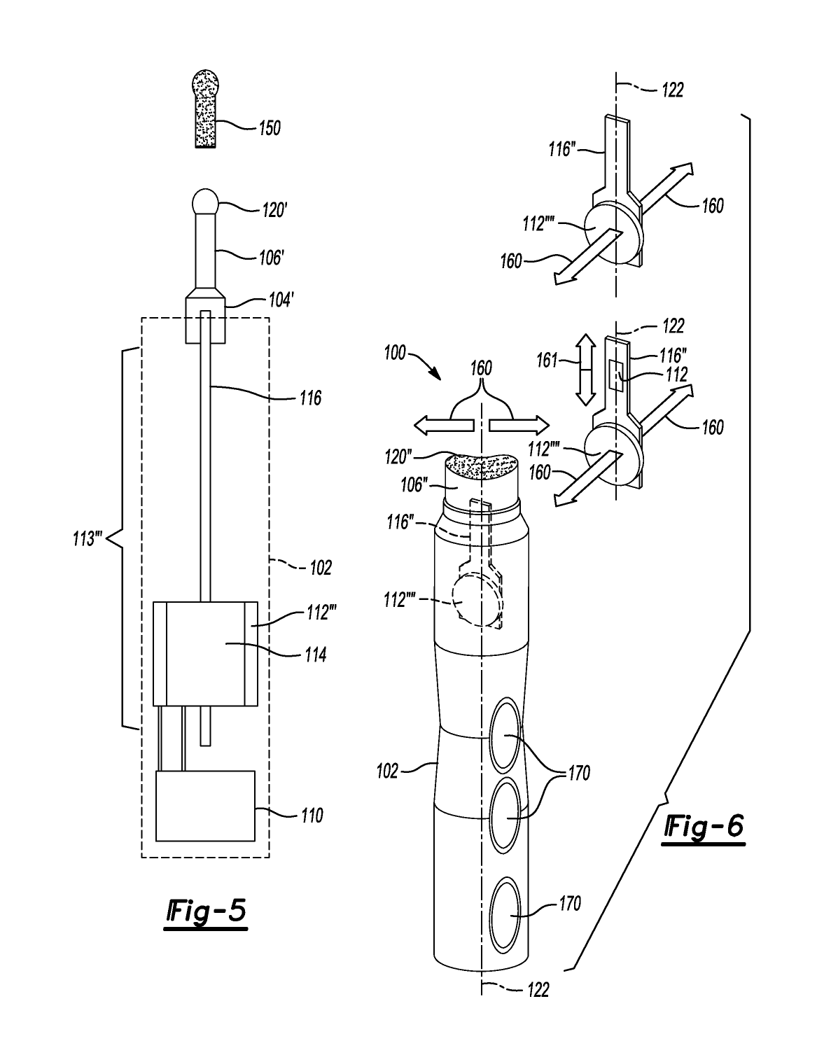

FIG. 5 depicts a drawing of the configuration of a piezoelectric cylinder 112′″ attached to the electric motor 114 within the body 102 of the ultrasonic eye cleaning device 100 according to an embodiment. The drive unit system 113′″ may include, but is not limited to, the piezoelectric cylinder 112′″ attached to the electric motor 114. The drive unit system 113′″ may receive operational instructions from the controls 110 based on operator input. The controls may be in communication with one or more selectable options at the user interface 202.

The ultrasonic eye cleaning device 100 as shown in FIG. 5 provides a fixed instrument 106 attached to the output member 116. The fixed instrument 106′ may be comprised of stainless steel or titanium. The drive unit system 113′″ may generate an oscillation and/or rotation to the fixed instrument 106′. The fixed instrument may allow for the open cell foam tip 150 to be applied for use during operation of the ultrasonic eye cleaning device 100. The open cell foam tip 150 may provide a disposable swab that may transmit the oscillation and/or rotation motion of the instrument 106′ and tip 120′. Once the treatment/operation to remove the debris from the eye is complete, the operator may remove and dispose of the open cell foam tip 150.

FIG. 6 depicts a drawing of a piezoelectric motor 112″″ within the body 102 of the ultrasonic eye cleaning device 100 according to an embodiment. The piezoelectric motor 112″ is assembled to an output member 116″. The piezoelectric motor 112″″ may generate an oscillation through the output member 116″ to allow the instrument 106″ and tip 120″ to move in a transverse oscillation 160 and/or an axial oscillation 161 along the center axis 122.

The instrument 106″ provides a large disc shaped cleaning pad which may alternately be attached to the body 102 of the device 100. The instrument 106″ is driven by the piezoelectric motor 112″″ generating a transverse oscillation at the tip pad 120″ to enable an operator to use the device on him/herself to clean lashes and/or eyelids. The ultrasonic eye cleaning device 100 may have one or more finger locators indented 170 on the body 102 to assist with the self-cleaning of debris from an operators eyelashes and/or eyelids

The instrument 106″ may transmit motion from the piezoelectric motor 112″″ configured with the output member 116″ to the tip 120″. The instrument 106″ may be a size sufficient to remove debris on the eyelid and/or eyelashes while transmitting the ultrasonic frequency to the tip 120″. For example, the instrument 106″ may have an approximate width of 10-30 millimeters. The tip 120″ design may also include, but is not limited to a configuration as a flat surface, a concave shape surface, or a rounded shape surface across the instrument 106″. In another embodiment, the tip 120″ design may be configured as a permeable shape across the instrument 106″. The tip 120″ may be a size sufficient to remove debris and transmit ultrasonic frequency on the eyelid and/or eyelashes. For example, the tip 120″ may have an approximate width of 10-30 millimeters. The tip 120″ may be configured to receive the open cell foam tip cover which is disposable after treatment of the eyelid and/or eyelashes.

In another embodiment, the output member may include one or more piezoelectric motors configured on the output member 116″ to generate both axial oscillation and transverse oscillation of the tip 120″. For example, two piezoelectric motors 112, 112″″ may be placed on the output member. In another example, a custom design piezoelectric disk may be configured on the output member 116″ to the movement of both axial and transverse oscillation of the tip 120″.

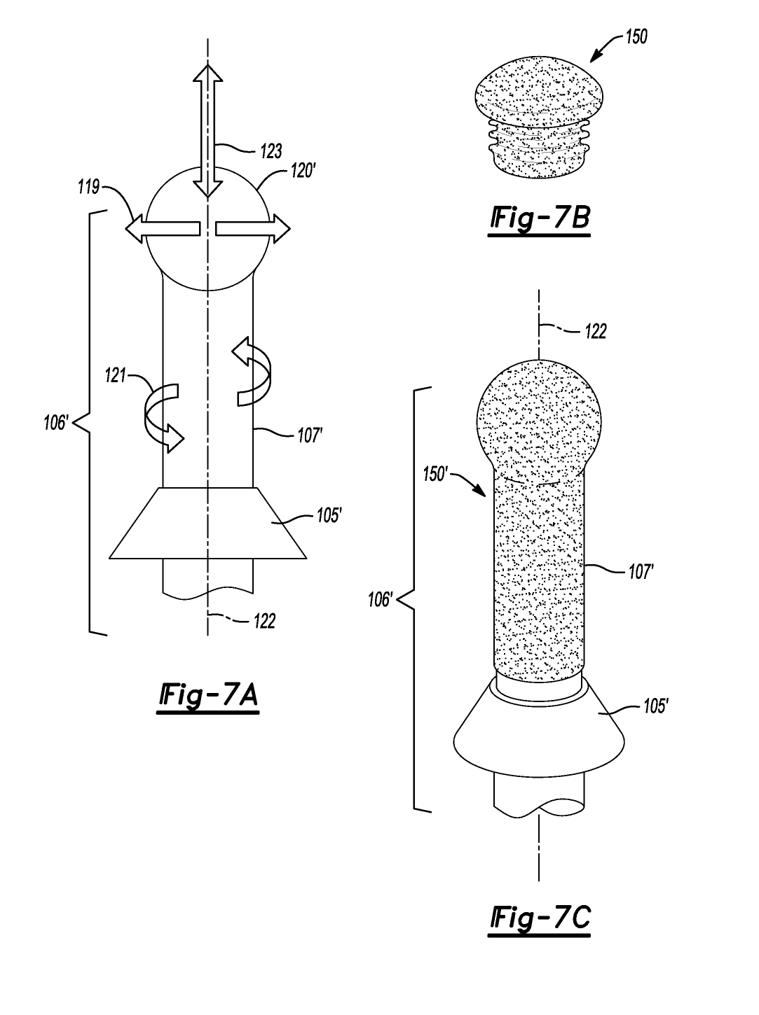

FIG. 7A-7C depict drawings of an instrument 106′ having at least one of a rotating and oscillating tip 120′ of the ultrasonic eye cleaning device 100 and a disposable cleaning swab according to an embodiment. The instrument 106′ comprises a tip portion 120′ and a shaft 107′ having a proximal end portion 105′ that is affixed to the body 102 of the device 100. The instrument 106′ may receive rotational and/or oscillating motion from the drive unit system 113 of the device 100.

FIG. 7A depicts a drawing of the instrument 106′ illustrating the oscillating motion in the axial direction 123 and the lateral direction 119 (i.e. transverse oscillation) along the center axis 122. The instrument 106′ may also rotate 121 in a forward and reverse direction while oscillating. The shaft 107′ is designed to have a length and width that is sufficient to transmit the oscillation and/or rotational motion from the drive unit system to the tip portion 120′ for breaking and/or removing debris on the eye. The tip portion 120′ is designed to have a length and width that is sufficient to receive the oscillation and/or rotation motion from the shaft 107′ to break and/or remove debris on the eye.

FIG. 7B depicts a drawing of the disposable cleaning swab in the form of an open cell foam tip 150 according to an embodiment. The open cell foam tip may be packaged in several forms including, but not limited to, a rolled-up form. An operator and/or a user may apply the open cell foam tip to the tip portion 120 and unroll the open cell foam tip 150 over the instrument 106.

FIG. 7C depicts a drawing of the instrument 106′ receiving the open cell foam tip 150 according to an embodiment. The open cell foam tip 150 may be configured to firmly cover the instrument 106′. Once secured to the tip 120′ and/or instrument 106′, the open cell foam tip 150′ is configured to simulate the oscillating and/or rotating motion of the instrument 106′ and tip 120′. The open cell foam tip 150 comprises a material that is of medical grade or surgical grade capable of breaking loose and/or removing debris on an eye without harming the eye. The open cell foam tip 150 is configured to transmit the ultrasonic frequency generated by the piezoelectric motor 112 to the eye.

The open cell eye foam tip 150′ may be removed from the instrument 106′ after an operator has completed treatment of the eye. The instrument 106′ may receive additional open cell eye foam tip(s) 150′ to remove debris on the eye.

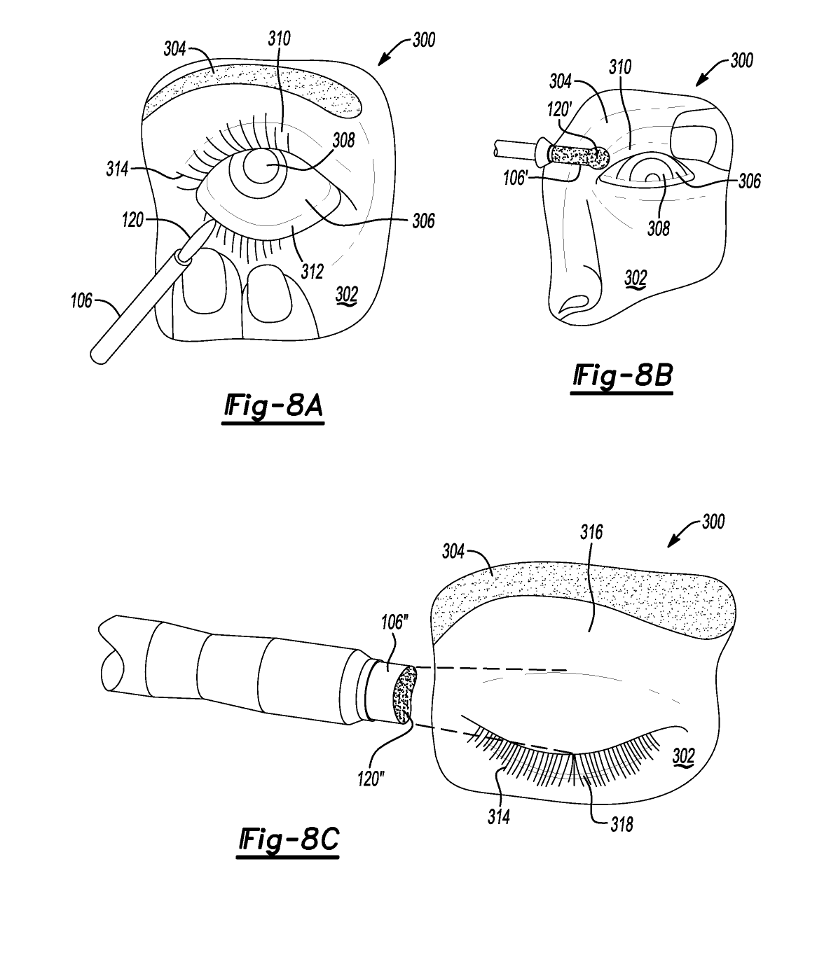

FIG. 8A-8C depict drawings of the device 100 used in a method for treating ocular disorders of the eye. FIGS. 8A and 8B generally show a portion of a face 302 having an eyebrow 304 and the eye 300. The eye 300 generally includes, but is not limited to, an eyeball 306 having a cornea 308, an upper eyelid 310, a lower eyelid 312, and a plurality of eyelashes 314. The ultrasonic eye cleaning device 100 comprises a drive unit system 113 to operably connect to the instrument 106 to allow oscillation and/or rotation of the tip portion 120. The ultrasonic oscillation and/or rotation of the tip portion 120 of the instrument 106 removes debris on the eye including the eyelid and eyelashes.

FIGS. 8A and 8B depict drawings of the rotating and/or oscillating tip 120 of the device 100 treating ocular disorders on the eye 300. The rotating and/or oscillating tip 120 may clean/treat the eyelashes 314, the upper inner eyelid 310, and the inside lower lid 312 including the margin. The device 100 may be powered on and set to a desirable speed by the operator. The desirable speed may include, but is not limited to, an amount of ultrasonic energy delivered, rotational/spinning speed, direction of the rotation/spinning of the tip 120, oscillation speed, and/or a combination thereof. The operator effects movement of the tip portion 120 relative to the ultrasonic eye cleaning device 100. The tip portion 120 is positioned near the eyeball 306 and along either one of the upper or lower eyelid margins 310, 312 for treatment.

For example, the operator may be treating a patient with an ocular disorder. The ocular disorder may include, but is not limited to, blepharitis. Before beginning the treatment of the ocular disorder, the operator may either prepare the device 100 by installing a new open cell eye foam tip 150 covering the instrument 106, or replacing the instrument with a new disposable shaft 107/tip 120 combination. The operator may target the debris on the eye 300 and eyelashes 310, 312 with the tip portion 120 of the device 100. The debris may be targeted by visually inspecting the eye 300 including the eyelids 310, 312 and the eyelashes 314 with or without the aid of a magnification device. Once the debris is identified and targeted by the operator, the tip portion 120 makes contact with that portion of the eye 300. The operator may instruct the patient to direct the cornea 308 away from the position of the tip portion 120 to minimize contacting the tip to the cornea 308 during treatment. For example, while treating the lower eyelid 312, the eyeball 306 directs the cornea 308 upward. While treating the upper eyelid 310, the eyeball 306 directs the cornea 308 downward. The operator may treat the eye 300 including the eyelids 310, 312, and eyelashes 314 by having the tip portion 120 make contact with the debris such that the oscillation breaks it up while the spinning/rotation removes the debris. The debris may be removed from the upper eyelid 310, lower eyelid 312, and/or from the plurality of eyelashes 314. The ultrasonic frequency may exfoliate the eyelids and eyelashes while eliminating the bacteria along the eyelid margins, as well as decreasing the number of any living organisms that may reside on the eyelid of in the eyelash follicle.

In another example, the operator may apply a liquid solution to the eye 300 to assist with the loosening of the debris. While breaking up and/or removing the debris, the tip portion 120 may absorb the liquid solution to assist in cleaning the eye 300. The liquid solution includes any solution that is capable of loosening the debris to further aid in removing the debris on the eye 300. The operator may apply the forward direction of the tip 120 rotation while moving the tip 120 from left to right across the upper and lower eyelids 310, 312. The operation may apply the reverse direction of the tip 120 rotation while moving the tip 120 from left to right across the upper and lower eyelids 310, 312.

The operator may use a finger, Q-tip, or similar gripping device, to move or lift a portion of the eye 300 to improve access to the debris located in the upper and lower eyelids 310, 312. The oscillation generated by the piezoelectric motor 112 at the tip portion 120 of the device 100 may resonate sound waves into the skin, creating a cavitation effect. The cavitation creates microscopic bubbles which may burst simultaneously releasing tiny amounts of energy in the skin.

FIG. 8C depicts the ultrasonic eye cleaning device with the pad shaped instrument 106″ treating ocular disorders of the eye 300. The fixed wand ultrasonic eye cleaning device may include the piezoelectric motor 112″″ configuration with the output member 116″ as shown in FIG. 6. The instrument 106″ is affixed to the body 102 of the device 100. The oscillating tip portion 120″ may clean/treat the outer upper and lower eyelids 316, 318 and the plurality of eyelashes 314. The oscillating tip 120″ may treat the ocular disorder by rubbing over the entire eyelid 316 to remove debris. The oscillating tip 120″ transmitting ultrasonic frequency may breakup and remove the debris, oil, and scurf that have been collected along the eyelid 316 during progression of the disorder. The oscillating tip portion 120″ transmitting ultrasonic frequency my treat conditions of the eye, such as Meibomian Gland Dysfunction by utilizing sources of ultrasonic treatment energy to implement heat and stimulation effects resulting in the release of lipids which may cause or be responsible for clogging of the meibomian gland. The sources of the ultrasonic treatment energy can be directed onto the upper and lower eyelids 316, 318 and specifically the meibomian glands located in the upper and lower eyelids 316, 318, and/or the surrounding soft tissue of the eyeball orbit. The source of treatment energy can comprise a source of ultrasonic energy into the meibomian glands of the eye 300 including the eyelids 316, 318. Introduction of the treatment energy into the meibomian glands may include an increase or facilitate an increase in heat of the surrounding orbital tissue, thereby mitigating the effects of meibomian gland syndrome. The oscillating tip portion 120″ transmitting ultrasonic frequency may also decrease the number of any living organisms that may reside on the eyelid and in or on the eyelash or eyelash follicle.

The fixed wand ultrasonic eye cleaning device may be operated by a patient for home treatment. The patient may treat his or her ocular disorder with the ultrasonic eye cleaning device 100 in periodic intervals. For example, the ultrasonic frequency being generated by the piezoelectric motor 112 may remove debris, exfoliate the eyelid, decrease the number of bacteria on the eyelid and eyelashes, and/or decrease the number of any living organisms (e.g., mites) that may reside on the eyelid or in the eyelash follicle of the patient's eyelid.

The method of treating the ocular disorder with the device 100 in treatments occurring in the periodic intervals achieves superior removal of the debris while eliminating bacteria compared to traditional treatments. The ultrasonic frequency and the oscillation of the tip 120 improve the treatment of drugs to the ocular disorder of the eye by causing sonophoresis. The sonophoresis of the eyelid is caused by the delivery of ultrasound to the eyelid's skin cells. The device 100 allows the physician and/or patient to treat the ocular disorder until the ocular disorder is sufficiently healed and thereafter to prevent a recurrence of the disorder. The ultrasonic eye cleaning device 100 may reduce inflammation and/or discomfort related to the debris within the eye 300. After each treatment, the device may be configured to remove the used instrument 106 from the chuck 104 and dispose of the used instrument. In another example, after each treatment, the open cell foam tip 150 may be removed from the instrument 106′/tip 120 and disposed of.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, embodiments described as less desirable than other embodiments or prior art implementations with respect to one or more characteristics are not outside the scope of the disclosure and can be desirable for particular applications.

1. An ultrasonic eye cleaning device comprising:
a body having an internal cavity and an end;
a user interface operable connected to one or more circuits within the internal cavity;
an instrument that is attachable to the end of the body and defining a free end forming a cleaning swab; and
a piezoelectric driver mounted in a manner to exercise the cleaning swab based on input received at the user interface.

2. The ultrasonic eye cleaning device of claim 1,
wherein the cleaning swab oscillates in an axial direction.

3. The ultrasonic eye cleaning device of claim 1, wherein the cleaning swab oscillates in a traverse direction.

4. The ultrasonic eye cleaning device of claim 1, wherein the piezoelectric driver, when energized, oscillates the cleaning swab at 20 kHz to 100 MHz.

5. The ultrasonic eye cleaning device of claim 1, wherein the instrument is affixed to the body.

6. The ultrasonic eye cleaning device of claim 5, wherein the instrument receives a disposable cell foam tip over the cleaning swab.

7. The ultrasonic eye cleaning device of claim 6, wherein the disposable cell foam tip is made of a material suitable to remove debris from an eyelid.

8. The ultrasonic eye cleaning device of claim 1, wherein the instrument is removable.

9. The ultrasonic eye cleaning device of claim 8, wherein the instrument has a disposable cleaning swab.

10. The ultrasonic eye cleaning device of claim 1, wherein the piezoelectric driver generates oscillation to at least one of the instrument and the cleaning swab.

11. An eye cleaning device comprising:
an elongate body having an internal cavity and an open end;
an output member having a base provided within the internal cavity and a shaft adjacent to the open end;
an instrument positioned at the open end of the body and attached to the shaft of the output member;
a first piezoelectric driver mounted in a manner to exercise the instrument, the piezoelectric driver, when energized, oscillates the instrument at 20 kHz to 100 MHz; and
a pad attached to the instrument to be oscillated by the piezoelectric driver for cleaning an eyelid.

12. The eye cleaning device of claim 11, wherein the pad is oscillated in a transverse axial direction relative to the elongate body.

13. The eye cleaning device of claim 11, wherein the pad is oscillated in an axial direction relative to the elongate body.

14. The eye cleaning device of claim 11, wherein the pad is made of a material suitable to remove debris from an eyelid.

15. The eye cleaning device of claim 11, further comprising, a second piezoelectric driver attached to the shaft of the output member to oscillate the pad in a transverse direction, and the first piezoelectric driver oscillates the pad in an axial direction relative to the elongate body.

16. The eye cleaning device of claim 11, wherein the pad has a concave shape surface across the instrument.

17. The eye cleaning device of claim 11, wherein the pad has a permeable shape surface across the instrument.

18. The eye cleaning device of claim 11, wherein the pad has a rounded shape surface across the instrument.

19. The eye cleaning device of claim 11, wherein the pad receives a disposable cell foam tip made from a material suitable to remove debris from an eyelid.

20. An eye cleaning method to treat an eye having an ocular disorder using an eye cleaning device, the method comprising:
generating an ultrasonic oscillation of an instrument having a free end forming a cleaning swab, the instrument affixed to an ultrasonic eye cleaning device;
providing the ultrasonic oscillation to the free end causing the cleaning swab to oscillate from 20 kHz to 100 MHz;
moving the instrument relative to the ultrasonic eye cleaning device; and
while the cleaning swab is being moved by the ultrasonic eye cleaning device, contacting a portion of an eyelid that includes debris based on the ocular disorder with the cleaning swab thereby impacting the debris with the ultrasonic oscillation with the swab and removing the debris from the eyelid.

21. The method of claim 20, further comprising removing the debris from the eyelid based on a rotational motion of the cleaning swab.

22. The method of claim 20, wherein the ultrasonic oscillation of the cleaning swab generating spinning water molecules in the eyelid of an eye and generating an exfoliation of the eyelids and eyelashes of the eye through cavitation.

23. The method of claim 20, wherein the ultrasonic oscillation of the cleaning swab is eliminating viruses in the eyelid of an eye by opening tiny pathways around cells based on vibrations of the cleaning swab.

24. The method of claim 20, wherein the ultrasonic oscillation of the cleaning swab is eliminating microscopic living organisms on the eyelid.

25. The method of claim 20, wherein the ultrasonic oscillation of the cleaning swab is heating the tissues through expansion, contraction, and collapse of gas in body fluids and tissue.

26. The method of claim 20, wherein the ultrasonic oscillation of the cleaning swab is generating sonophoresis delivery of bioactive compounds with direct targeting to the portion of the eyelid that is inflamed.

27. The ultrasonic eye cleaning device of claim 1, further comprising:
a motor oriented within the internal cavity, the motor having a stationary member and a rotating output member; and
a chuck driven by the rotating output member, wherein the instrument is attachable to the chuck.

28. The ultrasonic eye cleaning device of claim 27, wherein the cleaning swab oscillates in at least one of a radial direction.

29. The ultrasonic eye cleaning device of claim 1, wherein the body is an elongated body aligned along a central axis and the piezoelectric driver moves the cleaning swab in at least an axial direction.

30. The ultrasonic eye cleaning device of claim 29, wherein the cleaning swab is in a form of a disc oriented generally transversally to the central axis.

31. The ultrasonic eye cleaning device of claim 30, wherein the disc forms at least one of a concavely curve shape and a flat shape to cooperate with the eyelid of a user.

32. The method of claim 20, further comprising removing the debris from the eyelid based on an axial motion of the cleaning swab.

33. An eye cleaning device comprising:
a body having an internal cavity and an end;
a user interface located on the body and operable connected to one or more circuits within the internal cavity;
an instrument that is attachable to the end of the body and defining a free end forming a disc-shaped surface; and
a piezoelectric driver mounted in a manner to exercise the disc-shaped cleaning pad in an axial motion based on input received at the user interface.
